SEQUENCE LISTING (24 sequences)

SEQ ID NO: 1 - DNA, 18 bp, synthetic construct
  source 1..18: mol_type = other DNA
ggagtgtgagcaggggca

SEQ ID NO: 2 - DNA, 15 bp, synthetic construct
  source 1..15: mol_type = other DNA
cggctcctgctgccc

SEQ ID NO: 3 - DNA, 11 bp, synthetic construct
  source 1..11: mol_type = other DNA
  modified_base 1: mod_base = OTHER; note = 5'-6-Carboxyfluorescein-ctccgccag Zen quencher modified nucleotide
  modified_base 11: mod_base = OTHER; note = 3' Iowa Black FQ (IDT) quencher modified nucleotide
agtcggagccg

SEQ ID NO: 4 - DNA, 85 bp, Homo sapiens
  source 1..85: mol_type = genomic DNA
ggagtgtgagcaggggcaggagccaggagaagagcctccgccagagtcggagccggagcaggagcaaagggggcagcaggagccg

SEQ ID NO: 5 - DNA, 26 bp, synthetic construct
  source 1..26: mol_type = other DNA
accctgatttgctgctacatacttta

SEQ ID NO: 6 - DNA, 31 bp, synthetic construct
  source 1..31: mol_type = other DNA
catccattgatttataaactgtacagtgata

SEQ ID NO: 7 - DNA, 18 bp, synthetic construct
  source 1..18: mol_type = other DNA
  modified_base 1: mod_base = OTHER; note = 5'-6-Carboxyfluorescein-acgaattca Zen quencher modified nucleotide
  modified_base 18: mod_base = OTHER; note = 3' Iowa Black FQ (IDT) quencher modified nucleotide
gttgcctcacaaccctgg

SEQ ID NO: 8 - DNA, 18 bp, synthetic construct
  source 1..18: mol_type = other DNA
  modified_base 1: mod_base = OTHER; note = 5'-Hexachlorofluorescein-acgaattca Zen quencher modified nucleotide
  modified_base 18: mod_base = OTHER; note = 3' Iowa Black FQ (IDT) quencher modified nucleotide
gttgcctcacaaccctgg

SEQ ID NO: 9 - DNA, 89 bp, Homo sapiens
  source 1..89: mol_type = genomic DNA
accctgatttgctgctacatactttagaacgaattcagttgcctcacaaccctggacctatcactgtacagtttataaatcaatggatg

SEQ ID NO: 10 - DNA, 25 bp, synthetic construct
  source 1..25: mol_type = other DNA
ctatgaaaagtttggggagagtgag

SEQ ID NO: 11 - DNA, 21 bp, synthetic construct
  source 1..21: mol_type = other DNA
ctccgccttctcctgtttgtc

SEQ ID NO: 12 - DNA, 21 bp, synthetic construct
  source 1..21: mol_type = other DNA
  modified_base 1: mod_base = OTHER; note = 5'-6-Carboxyfluorescein-cctcatcag Zen quencher modified nucleotide
  modified_base 21: mod_base = OTHER; note = 3' Iowa Black FQ (IDT) quencher modified nucleotide
actcgacctccatctcaactt

SEQ ID NO: 13 - DNA, 82 bp, Homo sapiens
  source 1..82: mol_type = genomic DNA
ctatgaaaagtttggggagagtgaggaagttgagatggaggtcgagtctgatgaggaggatgacaaacaggagaaggcggag

SEQ ID NO: 14 - DNA, 20 bp, synthetic construct
  source 1..20: mol_type = other DNA
gtgacgacctccggatctga

SEQ ID NO: 15 - DNA, 19 bp, synthetic construct
  source 1..19: mol_type = other DNA
gccgagtagcagccactga

SEQ ID NO: 16 - DNA, 18 bp, synthetic construct
  source 1..18: mol_type = other DNA
  modified_base 1: mod_base = OTHER; note = 5'-Hexachlorofluorescein-cagggcgcg Zen quencher modified nucleotide
  modified_base 18: mod_base = OTHER; note = 3' Iowa Black FQ (IDT) quencher modified nucleotide
cgctcttatatagttcct

SEQ ID NO: 17 - DNA, 101 bp, Mus sp.
  source 1..101: mol_type = genomic DNA
gtgacgacctccggatctgagtccggattggctgtgagttcaggaactatataagagcgcgcgccctggtggctctctcatttcagtggctgctactcggc

SEQ ID NO: 18 - DNA, 737 bp, synthetic construct
  source 1..737: mol_type = other DNA
aaaagtcggagcaggagtcaggagaggagagtggaggaggagaagcgagggagtgtgagcaggggcaggagccaggagaagagcctccgccagagtcggagccggagcaggagcaaagggggcagcaggagccggactcgcaagctttctcaagcttcagctaggagactggtgacgacctccggatctgagtccggattggctgtgagttcaggaactatataagagcgcgcgccctggctggtctctcatttcagtggctgctactcggcgcttcagtcgcggtcgcttaagctttttagcaagcttcgtactatgtgtcttcaggttgcaattgctgccttgtactacaaccctgatttgctgctacatactttagaacgaattcagttgcctcacaaccctggacctatcactgtacagtttataaatcaatggatgaatgatacagattgttttcttgggcatcatgaccggaagatgtgtttgaagcttctagcaagcttttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagac

SEQ ID NO: 19 - DNA, 19 bp, synthetic construct
  source 1..19: mol_type = other DNA
gggttgcgctcgttgcagg

SEQ ID NO: 20 - DNA, 20 bp, synthetic construct
  source 1..20: mol_type = other DNA
cagatggtgcatggttgtcg

SEQ ID NO: 21 - DNA, 20 bp, synthetic construct
  source 1..20: mol_type = other DNA
gttactcacccattcgccgc

SEQ ID NO: 22 - DNA, 20 bp, synthetic construct
  source 1..20: mol_type = other DNA
gctggctgtgtgcctaatac

SEQ ID NO: 23 - DNA, 101 bp, Mus sp.
  source 1..101: mol_type = genomic DNA
gtgacgacctccggatctgagtccgattggctgtgagttcaggaactatataagagcgcgcgccctggctggctctcatttcagtggctgctactcggcgc

SEQ ID NO: 24 - DNA, 102 bp, Mus sp.
  source 1..102: mol_type = genomic DNA
gtgacgacctccggatctgagtccggattggctgtgagttcaggaactatataagagcgcgcgccctggctggctctcatttcagtggctgctactcggcgc

(19) United States
(12) Patent Application Publication
(10) Pub. No.: US 2024/0093308 A1
(43) Pub. Date: Mar. 21, 2024

(54) Human Fractional Abundance Assays

(71) Applicant: IDEXX Laboratories, Inc., Westbrook, ME (US)

(72) Inventors: Robert Schaad Livingston, Columbia, MO (US); Sarah Ann Hansen, Columbia, MO (US); Marcus Jeremy Crim, Columbia, MO (US)

(73) Assignee: IDEXX Laboratories, Inc., Westbrook, ME (US)

(21) Appl. No.: 18/362,238

(22) Filed: Jul. 31, 2023

Related U.S. Application Data
(60) Provisional application No. 63/394,152, filed on Aug. 1, 2022.

(51) Int. Cl. C12Q 1/6886; C12Q 1/686

(52) U.S. Cl. CPC C12Q 1/6886; C12Q 1/686; C12Q 2600/158; C12Q 2600/166

(57) ABSTRACT

Provided are assays that can provide the percentage of a first mammalian cell population (e.g., human cells) compared to a second mammalian cell population (e.g. mouse cells) in a mixed cell sample. The assays can be used to, for example, evaluate a wide variety of sample types, including humanized mice, humanized organs, passaged cell lines, and passaged tumors. These assays can be offered alone or in combination with, for example, cell line authentication and/or interspecies contamination testing.

### PRIORITY

This application claims the benefit of U.S. Ser. No. 63/394,152, filed on Aug. 1, 2022, which is incorporated by reference in its entirety herein.

### SEQUENCE LISTING

A sequence listing XML file is incorporated herein. The sequence listing is named 745081_IDX_013PC_SL.xml, was created on Oct. 2, 2023, and is 28,500 bytes in size.

### BACKGROUND

Researchers utilizing human-derived biologics require highly specific analyses to verify the molecular makeup of their samples at various timepoints throughout their studies. These diagnostic assays can reveal critical information regarding the source of the sample, evidence of contamination (or misidentification), and presence of genetic mutation or molecular infidelity over time. While current methods can be combined to develop a thorough understanding of the molecular makeup of these biologic samples, a challenge exists regarding evaluation of mixed-species populations, or specifically samples where human and murine cells coexist. This human/mouse mixture of cells is the most common type of contamination found in biologic samples, simply because of the tremendous value obtained from two specific model systems: the humanized mouse and the immune-deficient tumor-bearing mouse, including tumors derived from established neoplastic cell lines and patient-derived xenograft (PDX) models. These two model systems specifically account for an ever-increasing field of biomedical research poised to advance cancer diagnostics and therapeutics. Through previous methods such as real-time PCR or Sanger sequencing, a general estimation could be made of the comparative amount of human tumor cells vs. mouse stromal support cells remaining in an implanted tumor, or an in vitro passaged cell line. Despite this, these methods either lack accuracy, are time-intensive, or too costly to be used routinely, highlighting the need for a comparative method with a high level of sensitivity, specificity, and precision. Knowing the makeup of the species-specific cellular population of transplantable tumors and cultured cells will allow researchers to better interpret experimental outcomes, make critical decisions about the efficacy of therapeutics, assess the growth or decline of tumor cell populations and presence/absence of metastasis, and allow selection of cell populations with the highest human cellular component for cryopreservation or passage. In humanized mouse models, this assay can determine the relative amount of human cells to assess the degree of humanization of mice.

### SUMMARY

Provided herein are methods of quantifying an amount of mouse cells and human cells in a cell sample. The methods can comprise:
(a) assembling a single polymerase chain reaction (PCR) assay comprising template nucleic acid molecules from the cell sample and reagents suitable for the amplification of: (i) nucleic acid molecules comprising one mouse locus, (ii) nucleic acid molecules comprising a first human locus, and (iii) nucleic acid molecules comprising a second human locus;
(b) partitioning the single PCR reaction assay into partitioned sections, wherein each partitioned section contains no target loci or a single locus, and wherein the locus can be amplified within the partitioned section;
(c) performing the single polymerase chain reaction assay;
(d) quantifying a number of partitioned sections having an amplification product corresponding to the mouse locus, an amplification product corresponding to the first human locus, and an amplification product corresponding to the second human locus; and
(e) determining a Poisson-modeled number of partitioned sections having an amplification product corresponding to the mouse locus, a Poisson-modeled number of partitioned sections having an amplification product corresponding to the first human locus, and a Poisson-modeled number of partitioned sections having an amplification product corresponding to the second human locus,
   thereby determining an amount of mouse cells and human cells in the cell sample.

In some aspects, where there is a discrepancy of at least about 10% between the Poisson-modeled number of partitioned sections having an amplification product corresponding to the first human locus and the Poisson-modeled number of partitioned sections having an amplification product corresponding to the second human locus, then a single polymerase chain reaction (PCR) assay can be assembled comprising template nucleic acid molecules from the sample and reagents suitable for the amplification of nucleic acid molecules comprising one mouse locus and a third human locus, the single PCR reaction assay can be partitioned into partitioned sections, wherein each partitioned section contains no target loci or most often a single locus, and wherein the locus can be amplified within the partitioned sections. From there, the single polymerase chain reaction assay can be performed; a number of partitioned sections having an amplification product corresponding to the one mouse locus and the third human locus can be quantified; a Poisson-modeled number of partitioned sections having an amplification product corresponding to the one mouse locus and the third human locus can be determined, and the number of partitioned sections from two out of the first human locus, the second human locus, and the third human locus in closest agreement can be averaged.

At least one of the human loci can be stable in cancer. the mouse locus can be from a beta-2 microglobulin (B2m) gene and the first human locus and the second human locus can be from a serine/arginine-rich splicing factor (SRFS4) gene, an importin 8 (IPO8) gene, or a splicing factor 3a protein complex (SF3A1) gene. The mouse locus can be from a B2m gene, the first human locus, the second human locus, and the third human locus can be from a SRFS4 gene, an IPO8 gene, or a SF3A1 gene. The reagents for the amplification of the mouse locus can comprise polynucleotides as set forth in SEQ ID NOs:14, 15, and 16, the reagents for the amplification of the first human locus can comprise polynucleotides as set forth in SEQ ID NO:1, 2, and 3, the reagents for the amplification of the second human locus can comprise polynucleotides as set forth in SEQ ID NOs:5, 6, 7, and 8, and/or the reagents for the amplification of the third human locus can comprise polynucleotides as set forth SEQ ID NO:14, 15, and 16. The SRFS4 gene can be detected with a labeled probe as set forth in SEQ ID NO:3; the IPO8 gene can be detected with labeled probes as set forth in SEQ ID NOs:7 and 8, the SF3A1 gene can be detected with a labeled probe as set forth in SEQ ID NO:12, and the B2m gene can be detected with a labeled probe as set forth in SEQ ID NO:16.

The methods can further comprise amplifying and detecting two or more human short tandem repeats (STRs) of microsatellite regions. The two or more human STRs can be detected in two or more loci comprising D3S1358, TH01, D21S11, D18S51, Penta E, D5S818, D13S317, D7S820, D16S539, CSF1PO, Penta D, Amelogenin, vWA, D8S1179, TPOX, FGA, D19S433, D2S1338, or combinations thereof. The methods can include additionally amplifying one or more Mycoplasma nucleic acid molecules.

Another aspect provides methods of quantifying an amount of mouse cells and human cells in a cell sample. The methods can comprise:
(a) assembling a single polymerase chain reaction (PCR) assay comprising template nucleic acid molecules from the sample and reagents suitable for the amplification of: (i) nucleic acid molecules comprising one mouse locus, (ii) nucleic acid molecules comprising a first human locus, (iii) nucleic acid molecules comprising a second human locus, and (iv) nucleic acid molecules comprising a third human locus;
(b) partitioning the single PCR reaction assay into partitioned sections, wherein each partitioned section contains no target loci or a single locus, and wherein the locus can be amplified within the partitioned section;
(c) performing the single polymerase chain reaction assay;
(d) quantifying a number of partitioned sections having an amplification product corresponding to the mouse locus, an amplification product corresponding to the first human locus, an amplification product corresponding to the second human locus; and an amplification product corresponding to the third human locus;
(e) determining a Poisson-modeled number of partitioned sections having an amplification product corresponding to the mouse locus, a Poisson-modeled number of partitioned sections having an amplification product corresponding to the first human locus, a Poisson-modeled number of partitioned sections having an amplification product corresponding to the second human locus, and a Poisson-modeled number of partitioned sections having an amplification product corresponding to the third human locus;
wherein if there is a discrepancy of at least about 10% between the Poisson-modeled number of partitioned sections having an amplification product corresponding to the first human locus and the Poisson-modeled number of partitioned sections having an amplification product corresponding to the second human locus, then determining a Poisson-modeled number of partitioned sections having an amplification product corresponding to the third human locus, and averaging the number of partitioned sections from two out of the first human locus, the second human locus, and the third human locus in closest agreement
   thereby determining an amount of mouse cells and human cells in the cell sample.

Other aspects provide a kit for determining the fractional abundance of human genomic nucleic acid molecules compared to mouse genomic nucleic acid molecules in a mixed cell sample. A kit can comprise at least three sets of nucleic acid probes, primers, or pair of primers, wherein a first set of the at least three sets of nucleic acid probes, primers, or pair of primers is capable of specifically amplifying and detecting hSRSF4 nucleic acid molecules in a biological sample, a second set of the at least three nucleic acid sets of probes, primers, or pair of primers is capable of specifically amplifying and detecting hIPO8 nucleic acid molecules in the biological sample, and a third set of the at least three nucleic acid sets of probes, primers, or pair of primers is capable of specifically amplifying and detecting mB2m nucleic acid molecules in the biological sample. A kit can further comprise one or more of blocking agents, detectable labels or labeling agents, and reagents for hybridization. A kit can further comprises a fourth set of nucleic acid probes, primers, or pair of primers, wherein the fourth set of the at least three sets nucleic acid probes, primers, or pair of primers is capable of specifically amplifying and detecting hSF3A1 nucleic acid molecules in a biological sample. The at least three sets of nucleic acid probes, primers, or pair of primers are appropriate for use in digital PCR or droplet digital PCR (ddPCR). The at least three sets of nucleic acid probes, primers, or pair of primers can be detectably labeled. The first set of nucleic acid probe, primer, or pair of primers, the second set of nucleic acid probe, primer, or pair of primers, and the third set of nucleic acid probe, primer, or pair of primers can each be labeled with different detectable labels. The first set of nucleic acid probe, primer, or pair of primers, the second set of nucleic acid probe, primer, or pair of primers, the third set of nucleic acid probe, primer, or pair of primers can each be labeled with different fluorophores. The first set of the at least three sets of nucleic acid probes, primers, or pair of primers can be capable of specifically amplifying and detecting hSRSF4 nucleic acid molecules in a biological sample, and can comprise nucleic acid molecules as set forth in SEQ ID NOs:1, 2, and 3. The second set of the at least three nucleic acid sets of probes, primers, or pair of primers can be capable of specifically amplifying and detecting hIPO8 nucleic acid molecules in the biological sample and can comprise nucleic acid molecules as set forth in SEQ ID NOs:5, 6, 7, and 8. The third set of the at least three nucleic acid sets of probes, primers, or pair of primers can be capable of specifically amplifying and detecting mB2m nucleic acid molecules in the biological sample and can comprise nucleic acid molecules as set forth in SEQ ID NOs:14, 15, and 16. The fourth set of nucleic acid probes, primers, or pair of primers can be capable of specifically amplifying and detecting hSF3A1 nucleic acid molecules in a biological sample and can comprise nucleic acid molecules as set forth in SEQ ID NO:10, 11, and 12.

Another aspect provides methods of quantifying an amount of mouse cells and human cells in a cell sample. The methods can comprise:
(a) assembling a single polymerase chain reaction (PCR) assay comprising template nucleic acid molecules from the cell sample and reagents suitable for the amplification of: (i) nucleic acid molecules comprising one mouse locus and (ii) nucleic acid molecules comprising a first human locus;
(b) partitioning the single PCR reaction assay into partitioned sections, wherein each partitioned section contains no target loci or a single locus, and wherein the locus can be amplified within the partitioned section;
(c) performing the single polymerase chain reaction assay;
(d) quantifying a number of partitioned sections having an amplification product corresponding to the mouse locus and an amplification product corresponding to the first human locus; and
(e) determining a Poisson-modeled number of partitioned sections having an amplification product corresponding to the mouse locus and a Poisson-modeled number of partitioned sections having an amplification product corresponding to the first human locus,
   thereby determining an amount of mouse cells and human cells in the cell sample.
   The human loci can be stable in cancer. The mouse locus can be from a beta-2 microglobulin (B2m) gene, the first human locus can be from a serine/arginine-rich splicing factor (SRFS4) gene, an importin 8 (IPO8) gene, or a splicing factor 3a protein complex (SF3A1) gene. The reagents for the amplification of the mouse locus can comprise polynucleotides as set forth in SEQ ID NOs:14, 15, and 16, and the reagents for the amplification of the first human locus can comprise polynucleotides as set forth in SEQ ID NO:1, 2, and 3, SEQ ID NOs:5, 6, 7, and 8, and/or the reagents as set forth SEQ ID NO:14, 15, and 16. The methods can further comprise amplifying and detecting two or more human short tandem repeats (STRs) of microsatellite regions. The two or more human STRs can be detected in two or more loci comprising D3S1358, TH01, D21S11, D18S51, Penta E, D5S818, D13S317, D7S820, D16S539, CSF1PO, Penta D, Amelogenin, vWA, D8S1179, TPOX, FGA, D19S433, D2S1338, or combinations thereof. One or more Mycoplasma nucleic acid molecules can also be amplified. The SRFS4 gene can be amplified with primers as set forth in SEQ ID NOs:1 and 2; the IPO8 gene can be amplified with primers as set forth in SEQ ID NOs:5 and 6, the SF3A1 gene can be amplified with primers as set forth in SEQ ID NOs:10 and 11, and the B2m gene can be amplified with primers as set forth in SEQ ID NOs:14 and 15. The SRFS4 gene can be detected with a labeled probe as set forth in SEQ ID NO:3; the IPO8 gene can be detected with labeled probes as set forth in SEQ ID NOs:7 and 8, the SF3A1 gene can be detected with a labeled probe as set forth in SEQ ID NO:12, and the B2m gene can be detected with a labeled probe as set forth in SEQ ID NO:16.

Therefore, provided herein are assays that can provide the percentage of a first mammalian cell population (e.g., human cells) compared to a second mammalian cell population (e.g., mouse cells) in a mixed cell sample. The assays can be used to, for example, evaluate a wide variety of sample types, including but not limited to humanized mice, humanized organs, passaged cell lines, and passaged tumors. These assays can be offered alone or in combination with, for example, cell line authentication and/or interspecies contamination testing.

### BRIEF DESCRIPTION OF THE DRAWINGS

FIG. 1 shows human gene target chromosomal location for SRSF4 (Ch.1), IPO8 (Ch 12), and SF3A1 (Ch 22). (Source: Ryan L. Collins (Ryanlcollins.com).

FIG. 2 shows the B2m gene in NOD/ShiLtJ, DBA/2J, FVB/NJ, A/J, C3H/HeJ, 129S1/SvlmJ, C57BL/6NJ and BALB/cJ mouse strains (SEQ ID NO:23). The one exception exists in the BALB/cJ having a single insertion point mutation (within the amplicon but not in a primer/probe binding site)(SEQ ID NO:24).

FIG. 3 shows a summary of the process of ddPCR. Template and restriction enzyme (RE) are added to master mix. Sample is then vortexed and partitioned into droplets prior to thermal cycling. Droplets are then measured for fluorescence and quantified, then subjected to data analysis.

FIG. 4 shows the HindIII restriction enzyme target site.

FIG. 5 shows results from a duplex assay. Distinct separation of FAM-positive human amplicon droplets (cluster in upper left (1)), HEX-positive mouse amplicon droplets (cluster in lower right (4)), double-positive droplets that contain both amplicons (upper right cluster (2)), and negative droplets (lower left cluster (3)) can be seen.

FIG. 6 shows results from a triplex assay. Distinct separation of amplicons was achieved. Cluster (1) are hSRSF4 amplicon droplets; cluster (2) are droplets containing both hSRSF4 and hSF3A1; cluster (3) are hSF3A1 droplets; cluster (4) are droplets containing both mB2m and hSRSF4; cluster (5) are mB2m droplets; cluster (6) are droplets containing both mB2m and hSF3A1; cluster (7) are droplets containing hSRSF4, hSF3A1, and mB2m droplets; cluster (8) are negative droplets.

### DETAILED DESCRIPTION

Provided herein are assays based on digital PCR, e.g. digital droplet PCR, whereby one sample is partitioned into multiple (tens of thousands) of reactions prior to performing end-point PCR, which results in an exceptional method to determine the abundance or amount of human cells in a mixed cell population. The methods do not require standard curves and the simple presence vs. absence of the target sequence in each digital PCR reaction results in a highly accurate and precise method to determine the exact number of copies of a genomic sequence contained within the original sample population. Where single copy genes are selected for analysis, comparative results can be equated to cellular presence, resulting in a highly sensitive assay to determine the ratio of the number of human and murine cells in the sample being analyzed. As the platform results utilize Poisson distribution, the absolute concentration of the target within the sample aliquot can be accurately calculated as copies/μL. This valuable information can then be added to other biologic characterization assays to provide greater resolution into processes within humanized and/or tumor-bearing mice.

A human fractional abundance assay can be a stand-alone assay or one component of a robust cell authentication program. IDEXX Laboratories, Inc. (Westbrook ME) utilizes short tandem repeat (STR) profiles of extracted nucleic acids from tumor fragments and cell lines to obtain a genetic profile and determine the genetic similarity of the tumor or cell line with the source, as well as multiplex species-specific PCR to access for interspecies contamination of either mouse, rat, human, canine, Chinese hamster or African green monkey cells. In cases where human and mouse are found to coexist within the sample (>99% of explanted tumors), a human fractional abundance assay as described herein will be applied to provide a specific percent of human cells present, detecting as little as 1% human cells. Likewise, cell lines that have been passaged through mice routinely contain mouse stroma, and may be overtaken by mouse lymphocytes, retaining very little of the original human-origin sample. Other applications of this assay include evaluation of the presence of human leukocytes, fibroblasts, connective tissue or other cell types within the circulating or in situ cellular population of humanized mice, and mouse organ screening for presence of human cells indicative of either orthotopic implant presence or metastasis of implanted tumors. A wide variety of matrices have been confirmed to function well on this assay, including whole blood, tumor fragments, cell pellets or cells in suspension, organs, and formalin-fixed paraffin-embedded (FFPE) tissue blocks.

### Digital PCR

Digital PCR (dPCR) platforms include microfluidic-chamber based dPCR (e.g., BioMark® dPCR, Fluidigm), micro-well chip-based dPCR (e.g., QuantStudio12k flex dPCR), 3D PCR (Life Technologies), and droplet-based ddPCR (e.g., QX100 and QX200, BioRad®; RainDrop, RainDance®). Digital PCR is a nucleic acid amplification and detection method that is based on the dilution of target template nucleic acid molecules into independent, non-interacting partitions. See, e.g., Sykes et al. (1992) BioTechniques 13: 444-449. Following Poisson statistics with high dilutions of nucleic acid molecule template, each reaction is independently tested for the presence of a nucleic acid molecule at single molecule sensitivity. Partitioning can occur on microtiter plates or microfabricated platforms.

Droplet digital PCR (ddPCR) systems (e.g. Bio-Rad QX200) can disperse template DNA randomly into emulsion droplets of equal volume. ddPCR systems use quenched fluorescently-labeled polynucleotide probes to hybridize to a region of interest. Upon PCR amplification, the 5′ exonuclease activity of the polymerase separates the fluorophore from the quencher and generates a fluorescent signal specific for the target. The fluorescence of these partitions can be individually measured after amplification to determine the presence or absence of template molecules. The use of different fluorescent dyes allows for the simultaneous normalization of one genomic DNA region of interest or locus against a reference amplicon in a single reaction.

In digital PCR, a sample containing nucleic acid molecules (e.g., cells, lysed cells, tissues, or other biological samples) are separated into a large number of partitions. Partitioning can be achieved using micro well plates, capillaries, emulsions, arrays of miniaturized chambers, or nucleic acid molecule binding surfaces. Separation of a sample can involve distributing any suitable portion including up to the entire sample among the partitions. Each partition includes a fluid volume that is isolated from the fluid volumes of other partitions. About 500, 1,000, 5,000, 10,000, 50,000, 100,000, 500,000, 1,000,000, 2,000,000, 5,000,000 10,000,000 or more partitions can be present. The partitions can be isolated from one another by a fluid phase, such as a continuous phase of an emulsion, by a solid phase, such as at least one wall of a container, other suitable methods, or a combination thereof. The partitions can comprise droplets disposed in a continuous phase, such that the droplets and the continuous phase collectively form an emulsion.

Partitions can be formed by any suitable method. For example, partitions can be formed with a fluid dispenser, such as a pipette, with a droplet generator, by agitation of the sample (e.g., shaking, stirring, sonication, etc.), and other suitable methods. Partitions can be formed serially, in parallel, or in batch. Partitions can have uniform volume or can have different volumes. Exemplary partitions having substantially the same volume are monodisperse droplets. Partitions can comprise an average volume of less than about 100, 10 or 1 μL, less than about 100, 10, or 1 nL, or less than about 100, 10, or 1 μL.

After separation of the sample, PCR is carried out in the partitions. The partitions can be used for performance of one or more reactions. One or more reagents can be added to the partitions after they are formed in order to render them competent for reaction. The reagents can be added by any suitable mechanism, such as a fluid dispenser or fusion of droplets.

After PCR amplification, nucleic acid molecules can be quantified by counting the partitions that contain PCR amplicons for the target polynucleotides. Partitioning of the sample allows quantification of the number of different molecules by assuming that the population of molecules follows a Poisson distribution. See, e.g., Hindson et al. (2011) Anal. Chem. 83(22):8604-8610; Pohl and Shih (2004) Expert Rev. Mol. Diagn. 4(1):41-47; Pekin et al. (2011) Lab Chip 11 (13): 2156-2166; Pinheiro et al. (2012) Anal. Chem. 84 (2): 1003-1011; Day et al. (2013) Methods 59(1):101-107.

### Gene Location and Information

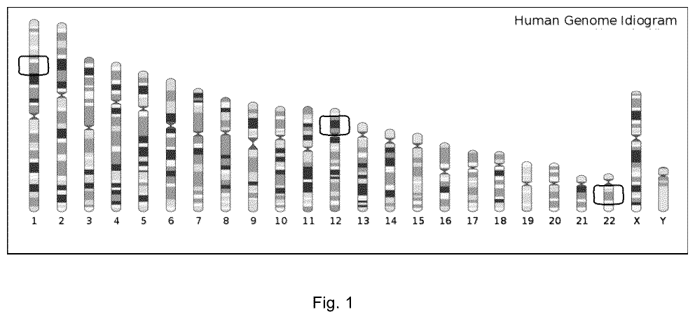

Digital PCR platforms such as droplet digital PCR can be used for detecting the human SRFS4 gene, the human IPO8 gene, the human SF3A1 gene, and/or the mouse B2m gene. This assay is extremely unique in that one, two, or three human genes are compared simultaneously to one mouse gene within the same sample aliquot. All human genes were selected to avoid genetic regions of questionable molecular fidelity due to the highly mutated neoplastic genomes exhibited in patient-derived xenografts (PDX), transplantable tumor models, and immortalized human cell lines. As splicing machinery is highly complex as well as utilized in neoplastic processes, many of pre-mRNA splicing genes appear to remain nearly unaffected in cancer. Selected human gene locations are shown in FIG. 1.

hSRFS4 encodes for serine/arginine-rich splicing factor 4, a gene that plays a role in alternative splice site selection during pre-mRNA splicing and represses the splicing of MAPT/Tau exon 10. hSRFS4 is located on human chromosome 1 and is 34,158 nucleotides in length, made up of 6 exons, translating to a protein of 494 amino acids. Both mouse and rat have orthologous genes: the mouse ortholog is Srsf4 (also known as Sfrs4), is located on chromosome 4, and is 28,182 nucleotides in length, translating to a protein of 368 amino acids.

hSF3A1 encodes for a subunit of the splicing factor 3a protein complex, which ultimately plays a critical role in spliceosome assembly and pre-mRNA splicing. It is located on human chromosome 22 and encodes for 793 amino acids.

hIPO8 encodes the protein importin 8 which functions in nuclear protein import. IPO8 is located on human Chromosome 12. The mouse has an orthologous gene, Ipo8 located on chromosome 6. The selected sequence for IPO8 is highly specific to humans and non-human primates and shares no matches with mouse or rat.

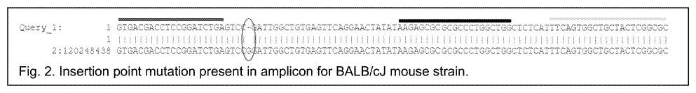

The promoter region of the mouse B2m gene (mB2m) was chosen as the mouse gene target and encodes for beta-2 microglobulin. The murine B2m gene is located on Chromosome 2 whereas the human orthologous B2M gene is located on Chromosome 15. mB2m was specifically screened utilizing NCBI Blast to ensure alignment in NOD/ShiLtJ, DBA/2J, FVB/NJ, A/J, C3H/HeJ, 129S1/SvlmJ, C57BL/6NJ and BALB/cJ mouse strains. The one exception exists in the BALB/cJ having a single point mutation (within the amplicon but not in a primer/probe binding site) as shown in FIG. 2.

### Fractional Abundance Assays

A fractional abundance assay can be performed on any biological matrix, cell population, or tissue population to obtain the percentage of human compared to mouse genomic nucleic acid molecules or cells present in the sample.

Provided herein are methods of quantifying a relative amount of mouse cells and human cells in a sample of cells. The methods can comprise assembling a single polymerase chain reaction (PCR) assay comprising template nucleic acid molecules from the sample and reagents suitable for the amplification of: (i) nucleic acid molecules comprising one mouse locus, (ii) nucleic acid molecules comprising a first human locus, (iii) optionally, nucleic acid molecules comprising a second human locus, and (iv) optionally, nucleic acid molecules comprising a third human locus. That is, one, two, or three human loci can be used. The single PCR reaction assay can be partitioned into partitioned sections, wherein each partitioned section contains no target loci or most often a single locus, and wherein the locus can be amplified within the partitioned section. The number of partitioned sections can be about 1,000, 5,000, 10,000, 20,000, 50,000, 100,000, 250,000, 500,000, 750,000, 1,000,000, 1,500,000, 2,000,000, 5,000,000, 10,000,000 or more. The number of partitioned sections having an amplification product corresponding to the mouse locus, an amplification product corresponding to the first human locus, and optionally, an amplification product corresponding to the second and/or third human locus are quantified. The positive droplet counts are then fit to a Poisson distribution model to account for the random distribution of the nucleic acid into discrete droplets. This model is applied to the number of partitioned sections having an amplification product corresponding to the mouse locus, the number of partitioned sections having an amplification product corresponding to the first human locus, and the number of partitioned sections having an amplification product corresponding to the second and/or third human locus are quantified such that the amount of mouse cells and human cells in the sample of cells are determined.

Where two human loci are used, if there is a discrepancy of at least about 5, 10, 20, 30, 40, 50, 60, 70% or more between the Poisson-modeled number of partitioned sections having an amplification product corresponding to the first human locus and the Poisson-modeled number of partitioned sections having an amplification product corresponding to the second human locus, then amplification products corresponding to third human locus can be used. The testing for the third human locus can be completed with (i.e., at the same time) the testing for the mouse locus, the first human locus, and the second human locus, or alternatively, can be completed separately. A single polymerase chain reaction (PCR) assay can be assembled comprising template nucleic acid molecules from the sample and reagents suitable for the amplification of nucleic acid molecules comprising a third human locus, partitioning the single PCR reaction assay into partitioned sections, wherein each partitioned section contains no target loci or most often a single locus, and wherein the locus can be amplified within the partitioned sections. A single polymerase chain reaction assay can be performed (i.e., thermocycling to amplify nucleic acids). A number of partitioned sections having an amplification product corresponding to the third human locus can be quantified. A Poisson-modeled number of partitioned sections having an amplification product corresponding to the third human locus can be determined. Then, the number of partitioned sections from two out of the three human loci (from the first human locus, the second human locus, and the third human locus) in closest agreement can be averaged and used to determine the amount of mouse cells and an amount of human cells in a sample of cells.

At least one of the three human loci (e.g., 1, 2, or 3) can be stable in cancer. The mouse locus can be from a beta-2 microglobulin (B2m) gene and the first human locus and the second human locus can be from a serine/arginine-rich splicing factor (SRFS4) gene, an importin 8 (IPO8) gene, or a splicing factor 3a protein complex (SF3A1) gene. The mouse locus can be from a B2m gene and the first human locus, the second human locus, and the third human locus can be from a SRFS4 gene, an IPO8 gene, or a SF3A1 gene.

Other suitable human loci include, for example, ACTB, YWHAZ, HPRT1, RNA18S, TBP, GAPDH, UBC, SNW1, CNOT4, HNRNPL, PCBP1, PPIA, PUM1, and RPL30. Other suitable mouse loci include, for example, Gapdh, Rn18s, Actb, Hprt, RpIpO, Gusb, and Ctbp1. These human and mouse loci demonstrate expression stability in cancer models.

The reagents for the amplification of the mouse locus can comprise polynucleotides as set forth in SEQ ID NOs:14, 15, and 16, the reagents for the amplification of the first human locus can comprise polynucleotides as set forth in SEQ ID NO:1, 2, and 3, the reagents for the amplification of the second human locus can comprise polynucleotides as set forth in SEQ ID NOs:5, 6, 7, and 8, and/or the reagents for the amplification of the third human locus can comprise polynucleotides as set forth SEQ ID NO:14, 15, and 16.

The SRFS4 gene can be amplified with primers as set forth in SEQ ID NOs:1 and 2; the IPO8 gene can be amplified with primers as set forth in SEQ ID NOs:5 and 6, the SF3A1 gene can be amplified with primers as set forth in SEQ ID NOs:10 and 11, and the B2m gene can be amplified with primers as set forth in SEQ ID NOs:14 and 15. Any other suitable primers for these genes can also be used.

The SRFS4 gene can be detected with a labeled probe as set forth in SEQ ID NO:3; the IPO8 gene can be detected with labeled probes as set forth in SEQ ID NOs:7 and 8, the SF3A1 gene can be detected with a labeled probe as set forth in SEQ ID NO:12, and the B2m gene can be detected with a labeled probe as set forth in SEQ ID NO:16. Any other suitable probes for these genes can also be used.

The method can further comprise verifying the identity of or establishing a genetic profile of the cells in the sample using a cell line authentication assay, (e.g., CellCheck, IDEXX Laboratories, Inc., Westbrook ME). A cell line authentication assay can comprise, for example, amplifying DNA hypervariable regions within genomes that makes it possible to identify human cell lines derived from a single donor. Hypervariable regions, which have variable number tandem repeat (VNTR) units from minisatellite DNA, can hybridize to many loci distributed throughout a genome to produce a DNA ‘fingerprint.” Short tandem repeats (“STRs”) of microsatellite regions have core sequences of 1-6 bp. The polymorphism or informativeness of these STR markers display many variations in the number of the repeating units between alleles and among loci in unrelated cell lines. Cell line authentication assays co-amplify and detect two or more (2, 4, 6, 8, 10, 12, 14, 16, 18 or more) of certain of these loci, e.g., D3S1358, TH01, D21S11, D18S51, Penta E, D5S818, D13S317, D7S820, D16S539, CSF1PO, Penta D, Amelogenin, vWA, D8S1179, TPOX, FGA, D19S433 and D2S1338. Four-color fluorescent detection or any other suitable detection method of the loci can be used. The results can be used to authenticate the origin of human cell lines, detect genetic drift, cell line contamination, and cell line misidentification. See, Reid et al. (Eds), Authentication of Human Cell Lines by STR DNA Profiling Analysis, In: Assay Guidance Manual [Internet]. Bethesda (MD): Eli Lilly & Company and the National Center for Advancing Translational Sciences; 2004-2013 May 1.

Additionally, one or more or more mycoplasma markers (e.g., 1, 2, 3, 4, 5, or more) can be assayed for contamination. The mycoplasma markers can be detected in the same fractional abundance assay or in a separate assay. For example, 70 bp and a 1062 bp mycoplasma-specific sequences of the 16S ribosomal RNA (rRNA) gene can be detected. See, e.g., U.S. Pat. Publ., which uses the following primers:

Mycoplasma-F1 GGGTTGCGCTCGTTGCAGG (SEQ ID NO:19); Mycoplasma-R1 CAGATGGTGCATGGTTGTCG (SEQ ID NO:20); Mycoplasma-F2 GTTACTCACCCATTCGCCGC (SEQ ID NO:21); Mycoplasma-R2 GCTGGCTGTGTGCCTAATAC (SEQ ID NO:22). F1 and R1 amplify a 70 base pair amplicon. Mycoplasma primers F1 and R2, also amplify a second mycoplasma-specific sequence of 1062 bp on the 16s rRNA gene. Any suitable mycoplasma marker genes and primers can be used. Mycoplasma markers can detect, for example, Mycoplasma arginini, Mycoplasma fermentans, Mycoplasma hominis, Mycoplasma hyorhinis, Mycoplasma pirum, Mycoplasma orale, Mycoplasma salivarium, Acholeplasma laidlawii, other Mycoplasma species, or combinations thereof.

Therefore, methods described herein can quantify an amount of mouse cells and an amount of human cells in a sample of cells and also authenticate the origin of human cell lines, detect genetic drift, cell line contamination, and cell line misidentification.

Samples can be cell populations such as mixed cell populations. Examples of samples included are cultured cells, an implantable tumor sample, a blood sample, humanized mouse tissue, mouse tissue, tumor tissue, humanized organ tissue, cell pellet, passaged cell lines, or other suitable cell population either fresh, refrigerated, frozen or formalin-fixed paraffin-embedded (FFPE). A cell population can be a non-naturally occurring cell population. A cell population can be an in vitro or ex vivo cell population.

In another method, quantifying an amount of mouse cells and human cells in a sample of cells can comprise assembling a single polymerase chain reaction (PCR) assay comprising template nucleic acid molecules from the sample and reagents suitable for the amplification of: (1) nucleic acid molecules comprising one mouse locus, (2) nucleic acid molecules comprising a first human locus, (3) nucleic acid molecules comprising a second human locus, and (4) nucleic acid molecules comprising a third human locus. The single PCR reaction assay can be portioned into partitioned sections, wherein each partitioned section contains no target loci or most often a single locus, and wherein the locus can be amplified within the partitioned section. The number of partitioned sections can be about 1,000, 5,000, 10,000, 20,000, 50,000, 100,000, 250,000, 500,000, 750,000, 1,000,000, 1,500,000, 2,000,000, 5,000,000, 10,000,000 or more. A single polymerase chain reaction assay can be performed (i.e., thermocycling to amplify nucleic acids).

A number of partitioned sections having an amplification product corresponding to the mouse locus, an amplification product corresponding to the first human locus, an amplification product corresponding to the second human locus; and an amplification product corresponding to the third human locus can be quantified.

Poisson distribution is applied and the Poisson-modeled number of partitioned sections having an amplification product corresponding to the mouse locus, the Poisson-modeled number of partitioned sections having an amplification product corresponding to the first human locus, the Poisson-modeled number of partitioned sections having an amplification product corresponding to the second human locus, and the Poisson-modeled number of partitioned sections having an amplification product corresponding to the third human locus can be determined.

Amplification products are distributed into partitioned sections such that each partition gets a number of molecules (0, 1, 2, etc.), theoretically following a Poisson distribution. Performing PCR on these partitions results in amplification being detected (positives) in those partitions containing one or more target molecules and no amplification being detected (negatives) in those partitions containing zero molecules. Since positive partitioned sections can contain more than one copy of the target molecule, a simple summing of the number of positive partitioned sections will not yield the correct number of target molecules present across the partitioned sections. Poisson distribution statistics are used to estimate the total number of target molecules present within the sample. The number of molecules per partitioned section is estimated from the fraction of partitioned sections not recording a molecule over an ensemble of partitioned sections. The estimate can then be divided by partition volume to obtain the Poisson-modeled number.

In a non-limiting example, after multiple PCR amplification cycles, the samples are checked for signal (e.g. fluorescence) with a binary readout of “0” or “1.” The fraction of total partitioned section with a detected target is recorded, which is equal to the number of partitioned sections in which target was detected divided by the total number of partitions. The partitioning of the sample allows estimation of the number of different molecules by assuming that the molecule population follows the Poisson distribution, thus accounting for the possibility of multiple target molecules inhabiting a single partition, Using Poisson's law of small numbers, the distribution of target molecule within the sample can be accurately approximated allowing for a quantification of the target in the PCR product. Poisson distribution of the copies of target molecule per droplet (CPD) based on the fraction of fluorescent droplets (p), is represented by the function CPD=−In(1−p). The model predicts that as the number of samples containing at least one target molecule increases, the probability of the sample containing more than one target molecule increases.

Therefore, the quantity of target sequences in a sample can be determined by assuming a mathematical correlation (e.g. Poisson's distribution) between the fraction of total number of partitioned sections wherein a target is identified and the number of target sequences per partitioned section; estimating the number of target sequences per partitioned section using the mathematical correlation; and calculating the quantity of target sequences in the sample by multiplying the number of target sequences per partitioned section and the total number of partitioned section, resulting in the Poisson-modeled number.

If there is a discrepancy of at least about 5, 10, 20, 30, 40, 50, 60, 70% or more between the Poisson-modeled number of partitioned sections having an amplification product corresponding to the first human locus and the Poisson-modeled number of partitioned sections having an amplification product corresponding to the second human locus, then determining a Poisson-modeled number of partitioned sections having an amplification product corresponding to the third human locus, and individually reporting or averaging the number of partitioned sections from two out of the three human loci in closest agreement thereby determining an amount of mouse cells and human cells in the cell sample.

### Polynucleotides, Probes, and Primers

Polynucleotides or nucleic acid molecules are a series of nucleotide bases: deoxyribonucleic acid (DNA) or ribonucleic acid (RNA). Nucleic acid molecules include but are not limited to genomic DNA, cDNA, mRNA, iRNA, miRNA, tRNA, ncRNA, rRNA, DNA-RNA hybrid sequences and recombinantly produced and chemically synthesized molecules such as aptamers, plasmids, antisense DNA strands, shRNA, ribozymes, nucleic acids conjugated, oligonucleotides or combinations thereof. Unless otherwise indicated, the term polynucleotide, nucleic acid molecule, or gene includes reference to the specified sequence as well as the complementary sequence thereof. Polynucleotides can be present as single-stranded or double-stranded and linear or covalently circularly closed molecule. As used herein, a polynucleotide can include both naturally occurring and/or non-naturally occurring nucleotides.

Polynucleotides can be obtained from nucleic acid molecules present in, for example, a mammalian cell. Polynucleotides can also be synthesized in the laboratory, for example, using an automatic synthesizer. Polynucleotides can be isolated. An isolated polynucleotide can be a naturally occurring polynucleotide that is not immediately contiguous with one or both of the 5′ and 3′ flanking genomic sequences with which it is naturally associated. An isolated polynucleotide can be, for example, a recombinant DNA molecule of any length, provided that the nucleic acid molecules naturally found immediately flanking the recombinant DNA molecule in a naturally occurring genome are removed or absent. Isolated polynucleotides also include non-naturally occurring nucleic acid molecules. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (vi) extracted from a sample.

Polynucleotides can encode full-length polypeptides, polypeptide fragments, and variant or fusion polypeptides. Polynucleotides can comprise coding sequences for naturally occurring polypeptides or can encode altered sequences that do not occur in nature. Polynucleotides can be purified free of other components, including, but not limited to proteins, lipids and other polynucleotides. For example, the polynucleotide can be 50%, 75%, 90%, 95%, 96%, 97%, 98%, 99%, or 100% purified. A polynucleotide existing among hundreds to millions of other polynucleotide molecules within, for example, cDNA or genomic libraries, or gel slices containing a genomic DNA restriction digest are not to be considered a purified polynucleotide.

In one aspect, a polynucleotide comprises a probe, primer, or amplicon as shown in SEQ ID Nos:1-24 In some aspects, a nucleic acid molecule comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequences set forth in SEQ ID Nos:1-24 or a fragment thereof. A fragment can be about 5, 10, 15, 20, 25, or more nucleotides. Programs such as OligoPerfect Primer Designer (ThermoFisher Scientific), Primer Express™ (ThermoFisher Scientific), PrimerQuest™ Tool (Integrated DNA Technologies), and/or similar software can be used to design primers and probes.

A primer includes all forms of primers including peptide nucleic acid primers, locked nucleic acid primers, phosphorothioate modified primers, labeled primers, and other suitable primers. Primers are typically at least about 10, 15, 20, 25, 30, 35, 40, 45, 50, or more nucleotides in length.

A probe is a nucleic acid molecule that can interact with a target nucleic acid molecule by hybridization. Probes can be, for example, polynucleotides, artificial chromosomes, fragmented artificial chromosomes, genomic nucleic acid molecules, fragmented genomic nucleic acid molecules, DNA, RNA, recombinant nucleic acid molecules, fragmented recombinant nucleic acid molecules, peptide nucleic acid (PNA) molecules, locked nucleic acid molecules, oligomers of cyclic heterocycles, or conjugates of nucleic acid molecules. Probes can comprise modified nucleobases and/or modified sugar moieties. A probe can be fully complementary to a target nucleic acid molecule or only partially complementary (e.g. about 70, 80, 90, 95, 98, 99%, or more homology). A probe can be used to detect the presence or absence of a target nucleic acid.

Primers and probes can be labeled with a detectable molecule or substance, such as a fluorescent molecule, a radioactive molecule, or any other suitable labels.

A label or detectable label is a moiety that can be attached to a primer or probe to render the primer or probe detectable, such as a moiety attached to a probe such that the probe can be detectable upon binding to a target sequence. In some embodiments, the moiety alone may not be detectable but can become detectable upon reaction with another moiety.

A detectable label can generate a signal such that the intensity of the signal is proportional to the amount of bound target. Labeled nucleic acid molecules can be prepared by incorporating or conjugating a label that is directly or indirectly detectable by spectroscopic, photochemical, biochemical, immunochemical, electrical, optical, chemical or other means. Suitable detectable labels include, for example, radioisotopes, fluorophores (e.g. fluorescein isothiocyanate (FITC), phycoerythrin (PE), cyanine (Cy3), VIC fluorescent dye, FAM (6-carboxyfluorescein) or Indocyanine (Cy5), chromophores, chemiluminescent agents, microparticles, enzymes, magnetic particles, electron dense particles, mass labels, spin labels, haptens, and other suitable labels). Probes and primers can be labeled by coupling or physically linking a detectable moiety or by indirect labeling by reactivity with another reagent that is directly labeled.

Many real-time detection chemistries can be used to indicate the presence of amplified nucleic acid molecules. Some detection chemistries depend upon fluorescence indicators that change properties as a result of the PCR process. Among these detection chemistries are DNA binding dyes (such as SYBR® Green) that increase fluorescence efficiency upon binding to double stranded DNA. Other real-time detection chemistries can be used including Foerster resonance energy transfer (FRET), where the fluorescence efficiency of a dye is strongly dependent on its proximity to another light absorbing moiety or quencher. These dyes and quenchers are typically attached to a probe or primer. Among the FRET-based detection chemistries are hydrolysis probes and conformation probes. Hydrolysis probes (such as the TaqMan® probe) use a polymerase enzyme to cleave a reporter dye molecule from 1, 2, or more quencher dye molecules attached to a polynucleotide probe. Conformation probes (such as molecular beacons) utilize a dye attached to a polynucleotide, whose fluorescence emission changes upon the conformational change of the polynucleotide hybridizing to the target DNA.

### Kits

Provided herein are kits for determining the fractional abundance of human genomic nucleic acid molecules compared to mouse genomic nucleic acid molecules in a mixed cell sample comprising at least three sets of nucleic acid probes, primers, or pair of primers. A first set of the at least three sets nucleic acid probes, primers, or pair of primers can be capable of specifically amplifying and detecting hSRSF4 nucleic acid molecules in a biological sample, a second set of the at least three nucleic acid sets of probes, primers, or pair of primers can be capable of specifically amplifying and detecting hSF3A1 nucleic acid molecules in the biological sample, and a third set of the at least three nucleic acid sets of probes, primers, or pair of primers can be capable of specifically amplifying and detecting mB2m nucleic acid molecules in the biological sample.

A kit can further comprise a fourth set of nucleic acid probes, primers, or pair of primers, wherein the fourth set of the at least three sets nucleic acid probes, primers, or pair of primers can be capable of specifically amplifying and detecting hIPO8 nucleic acid molecules in a biological sample.

A kit can further comprise one or more of blocking agents, detectable labels, or labeling agents, and reagents for hybridization.

The at least three sets of nucleic acid probes, primers, or pair of primers can be appropriate for use in digital PCR. The at least three sets of nucleic acid probes, primers, or pair of primers can be appropriate for use in droplet digital PCR (ddPCR).

The at least three sets of nucleic acid probes, primers, or pair of primers can be detectably labeled. The first set of nucleic acid probe, primer, or pair of primers, the second set of nucleic acid probe, primer, or pair of primers, and the third set of nucleic acid probe, primer, or pair of primers are each labeled with different detectable labels.

A kit can comprise a first set of nucleic acid probe, primer, or pair of primers, a second set of nucleic acid probe, primer, or pair of primers, and a third set of nucleic acid probe, primer, or pair of primers can each be labeled with different fluorophores.

A kit can comprise a first, second, and third set of nucleic acid probes, primers, or pairs of primers. A kit can comprise a first, second, third, and fourth set of nucleic acid probes, primers, or pairs of primers. A first set of nucleic acid probes, primers, or pair of primers can be capable of specifically amplifying and detecting hSRSF4 nucleic acid molecules in a biological sample. For example, one or more (1, 2, or 3) of nucleic acid molecules as set forth in SEQ ID NOs:1, 2, and 3. A second set of probes, primers, or pair of primers can be capable of specifically amplifying and detecting hSF3A1 nucleic acid molecules in a biological sample. For example, one or more (1, 2, 3, or 4) of nucleic acid molecules as set forth in SEQ ID NOs:10, 11, and 12. A third set of probes, primers, or pair of primers can be capable of specifically amplifying and detecting mB2m nucleic acid molecules in a biological sample. For example, one or more (1, 2, or 3) nucleic acid molecules as set forth in SEQ ID NOs:14, 15, and 16. A fourth set of nucleic acid probes, primers, or pair of primers can be capable of specifically amplifying and detecting hIPO8 nucleic acid molecules in a biological sample. For example, one or more (1, 2, or 3) nucleic acid molecules as set forth in SEQ ID NO:5, 6, 7, and 8.

A kit can comprise a set of nucleic acid probes, primers, or pair of primers that are capable of specifically amplifying and detecting hSRSF4 nucleic acid molecules in a biological sample. A kit can comprise a set of probes, primers, or pair of primers are capable of specifically amplifying and detecting hSF3A1 nucleic acid molecules in a biological sample. A kit can comprise a set of probes, primers, or pair of primers that are capable of specifically amplifying and detecting mB2m nucleic acid molecules in a biological sample. A kit can comprise a set of nucleic acid probes, primers, or pair of primers that are capable of specifically amplifying and detecting hIPO8 nucleic acid molecules in a biological sample.

A kit can comprise one, two, three, or four sets of nucleic acid probes, primers, or pair of primers that are capable of specifically amplifying and detecting hSRSF4, hSF3A1, mB2m, and/or hIPO8 nucleic acid molecules in any combination.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1

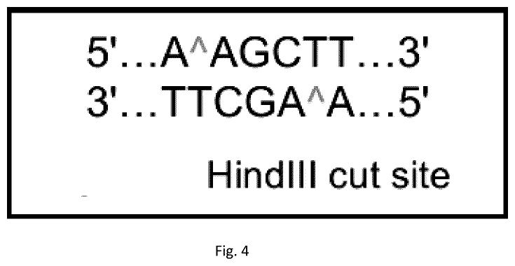

Restriction enzymes are used in ddPCR assays to allow for proper partitioning of sample within droplets prior to thermocycling. Restriction enzymes (RE) must be selected based on amplicon sequences, so as to not cut within an amplicon. The restriction enzyme utilized by the assays described herein is HindIII, as shown in FIG. 4, which has a starting concentration of 20,000 U/ml. In this assay, the goal is to obtain 15 U of RE in the final 20 μL reaction. This was done by adding 25 μL RE into 75 μL CutSmart® buffer to obtain 20 U/μL RE in a working stock. This was then added to each sample and allowed to incubate for 10 minutes at room temperature prior to droplet generation.

Reagents required for this assay include a supermix designed for hydrolysis-probe-based ddPCR, primers, single or double-quenched hydrolysis-based probes, restriction enzyme, water, and template. Acceptable template can be extracted nucleic acids using any variety of methods, either chemical-, kit- or liquid-handling-robotic-based extraction. Primers and probes are mixed according to the particular assay's validation methods as indicated in Table 1.

TABLE 1
Reagents required for duplex and triplex reactions.

| | | Volume |
|---|---|---|
| | | per 22 μL |
| Reagent | Concentration | reaction |
| 2X ddPCR Supermix | 1X | 11 |
| 20X primer/probe mix | 950 nM primer | 1 |
| FAM | 250 nM probe | |
| 20X primer/probe mix | 950 nM primer | 1 |
| HEX | 250 nM probe | |
| Diluted restriction enzyme | 1X | 1 |
| Template | variable | n |
| Water | | 8-n |
| Total | | 22 |
| 2X ddPCR Supermix | 1X | 11 |
| 20X primer/probe mix | 950 nM primer | 1 |
| FAM | 250 nM probe | |
| 20X primer/probe mix | 950 nM primer | 1 |
| HEX | 250 nM probe | |
| 20X primer/probe mix | 950 nM primer | 1 |
| HEX/FAM combo | 250 nM probe | |
| Diluted restriction enzyme | 1X | 1 |
| Template | variable | n |
| Water | | 7-n |
| Total | | 22 |

Prior to thermocycling and after template was added to master mix, samples were thoroughly vortexed. Samples were then partitioned into droplets by either robotic droplet generation or manually utilizing an 8-well droplet generation machine. Plates containing partitioned sample were then thermocycled according to the optimized parameters as shown in Table 2.

Thermal cycling parameters for each assay were determined through assay optimization experiments. Specifically, each assay was tested and developed in a single format including the following experiments: temperature gradient, probe concentration, assay specificity, assay sensitivity. Next, assays were combined to develop duplex and triplex assays. These assays were tested first with synthetic nucleic acids containing amplicon sequences separated by restriction enzyme cut sites. Assays were analyzed for ideal annealing temperature (Ta) and resolution. Resolution (ideal >1.5) is defined as the amplitude of the positive droplet population minus the amplitude of the negative population divided by the amplitude of the negative population. Optimal thermal cycling parameters for all assays are listed in Table 2.

TABLE 2
Optimal thermocycling parameters for all assays with human
fractional abundance testing. All steps are performed at
a 2.5° C./sec ramp rate with a lid temperature of 105° C.

| Thermal Cycling | | | |
|---|---|---|---|
| Step | Temp. ° C. | Time (min.) | |
| Hot Start/Initial | 95 | 10 | |
| Denaturation | | | |
| Denaturation | 95 | 0.5 | Repeat 39 times |
| Annealing | 63 | 1 | |
| Final | 98 | 10 | |
| Extension/Enzyme | | | |
| Deactivation | | | |
| Cooling | 4 | 30 | |
| Storage | 12 | ∞ | |

Individual assays and amplicons were designed based on specific thermocycling parameters during the development process. Final assays are outlined in Table 3.

TABLE 3
Primer and probe sequence for each assay utilized within the human
fractional abundance test.

| | Primer/Probe | | |
|---|---|---|---|
| Gene | name | Sequence | SEQ ID NO: |
| hSRSF4 | hSRSF4f | GGAGTGTGAGCAGGGGCA | SEQ ID NO: 1 |
| hSRSF4 | hSRSF4r | CGGCTCCTGCTGCCC | SEQ ID NO: 2 |
| hSRSF4 | hSRSF4- | /56-FAM/CTCCGCCAG/ZEN/ | SEQ ID NO: 3 |
| | FAM | AGTCGGAGCCG/3IABkFQ/ | |
| hIPO8 | hIPO8f | ACCCTGATTTGCTGCTACATACTTTA | SEQ ID NO: 5 |
| hIPO8 | hIPO8r | CATCCATTGATTTATAAACTGTACAGTGATA | SEQ ID NO: 6 |
| hIPO8 | hIPO8f-FAM | /56-FAM/ACGAATTCA/ZEN/GTT | SEQ ID NO: 7 |
| | | GCCTCACAACCCTGG/3IABkFQ/ | |
| hIPO8 | hIPO8f-HEX | /5-HEX/ACGAATTCA/ZEN/GTT | SEQ ID NO: 8 |
| | | GCCTCACAACCCTGG/3IABkFQ | |
| hSF3A1 | hSF3A1f | CTATGAAAAGTTTGGGGAGAGTGAG | SEQ ID NO: 10 |
| hSF3A1 | hSF3A1r | CTCCGCCTTCTCCTGTTTGTC | SEQ ID NO: 11 |
| hSF3A1 | hSF3A1- | /56-FAM/CCTCATCAG/ZEN/ | SEQ ID NO: 12 |
| | FAM | ACTCGACCTCCATCTCAACTT/3IABkFQ/ | |
| mB2m | mB2mf | GTGACGACCTCCGGATCTGA | SEQ ID NO: 14 |
| mB2m | mB2mr | GCCGAGTAGCAGCCACTGA | SEQ ID NO: 15 |
| mB2m | mB2mf-HEX | /5HEX/CAGGGCGCG/ZEN/CGC | SEQ ID NO: 16 |
| | | TCTTATATAGTTCCT/3IABkFQ/ | |

The 85 base pair amplicon for hSRSF4 is shown in SEQ ID NO:4

GGAGTGTGAGCAGGGGCAGGAGCCAGGAGAAGAGCCTCCGCCAGAGTCGG
AGCCGGAGCAGGAGCAAAGGGGGCAGCAGGAGCCG

The resolution is 12.7 (ideal is greater than 1.5).
The 89 base pair amplicon for hIPO8 is shown in SEQ ID NO:9

ACCCTGATTTGCTGCTACATACTTTAGAACGAATTCAGTTGCCTCACAAC
CCTGGACCTATCACTGTACAGTTTATAAATCAATGGATG

The resolution is 8.43 (ideal is greater than 1.5).
The 82 base pair amplicon for hSF3A1 is shown in SEQ ID NO:13

CTATGAAAAGTTTGGGGAGAGTGAGGAAGTTGAGATGGAGGTCGAGTCTG
ATGAGGAGGATGACAAACAGGAGAAGGCGGAG

The resolution is 3.76 (ideal is greater than 1.5).
The 101 base pair amplicon for mB2m is shown in SEQ ID NO:17

GTGACGACCTCCGGATCTGAGTCCGGATTGGCTGTGAGTTCAGGAACTA
TATAAGAGCGCGCGCCCTGGTGGCTCTCTCATTTCAGTGGCTGCTACTC
GGC

The resolution is 4.9 (ideal is greater than 1.5).
The synthetic sequence used for assay development and positive control is:

SEQ ID NO: 18
AAAAGTCGGAGCAGGAGTCAGGAGAGGAGAGTGGAGGAGGAGAAGCGAGG
GAGTGTGAGCAGGGGCAGGAGCCAGGAGAAGAGCCTCCGCCAGAGTCGGA
GCCGGAGCAGGAGCAAAGGGGGCAGCAGGAGCCGGACTCGCAAGCTTTCT
CAAGCTTCAGCTAGGAGACTGGTGACGACCTCCGGATCTGAGTCCGGATT
GGCTGTGAGTTCAGGAACTATATAAGAGCGCGCGCCCTGGCTGGTCTCTC
ATTTCAGTGGCTGCTACTCGGCGCTTCAGTCGCGGTCGCTTAAGCTTTTT
AGCAAGCTTCGTACTATGTGTCTTCAGGTTGCAATTGCTGCCTTGTACTA
CAACCCTGATTTGCTGCTACATACTTTAGAACGAATTCAGTTGCCTCACA
ACCCTGGACCTATCACTGTACAGTTTATAAATCAATGGATGAATGATACA
GATTGTTTTCTTGGGCATCATGACCGGAAGATGTGTTTGAAGCTTCTAGC
AAGCTTTTCCAACCCAATGAGCAAGGGAACTTCCCTCCCCCCACCACGCC
AGAGGAGCTGGGGGCCCGAATCCTCATTCAGGAGCGCTATGAAAAGTTTG
GGGAGAGTGAGGAAGTTGAGATGGAGGTCGAGTCTGATGAGGAGGATGAC
AAACAGGAGAAGGCGGAGGAGCCTCCTTCCCAGCTGGACCAGGACACCCA
AGTACAAGATATGGATGAGGGTTCAGATGATGAAGAC.

### Example 2

In some embodiments two or more human STRs can also be detected in the same fractional abundance assay or in a separate assay. Tumor samples were first analyzed for cell line authentication as follows: tumors/cells were processed for total nucleic acid (TNA) extraction. DNA was then subjected to a panel of human STR markers. These are specific core sequences of 1-6 base pairs that are highly variable in the human population and is used to authenticate the origin of human cell lines and detect genetic drift, cell line contamination, and cell line misidentification. CellCheck (IDEXX Laboratories, Westbrook ME) uses either 8 or 18 loci to evaluate human cell lines: D3S358, TH01, D21S11, D18S51, Penta E, D5S818, D13S317, D7S820, D16S539, CSF1PO, Penta D, amelogenin, vWa, D8S1179, TPOX, FGA, D19S433 and D2S1338. The primers utilized for this analysis are covalently linked to fluorescent molecules for PCR amplification. These amplicons were then detected with the Applied Biosystems Genetic Analyzer and determined to either match or show discrepancies in the expected outcome. During this analysis, any presence of mouse nucleic acids would have no effect on the cell line authentication and would also not be detected by CellCheck (STR testing). Therefore, combining the human:mouse fractional abundance assay with CellCheck (STR testing) provides critical additional information regarding percentage of the analyzed tumor or human cells present as compared to mouse stromal ingrowth.

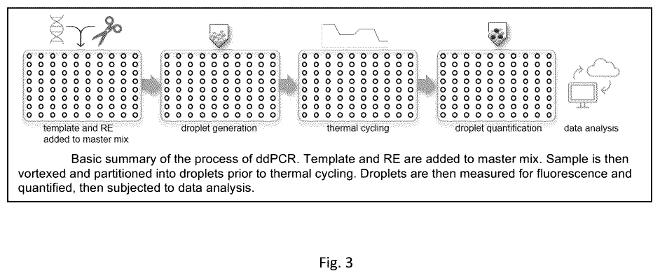

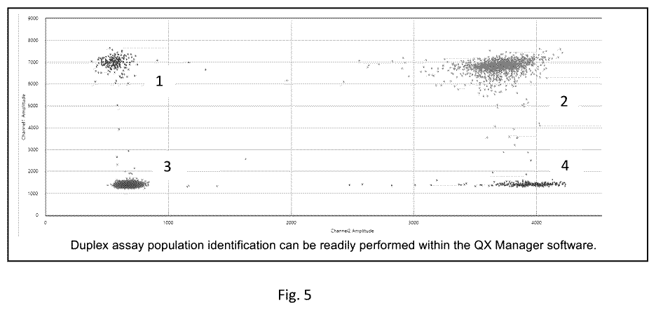

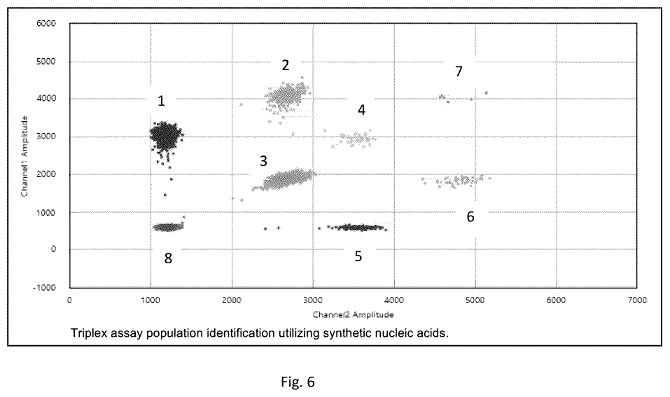

When the assay was performed in a duplex reaction, the BioRad QX Manager Software was relied upon to identify populations of interest to perform analysis as shown in FIG. 3 below. When a triplex assay (FIG. 6) is performed, analysis requires trained user input to identify populations, as indicated in FIG. 5. To simplify, all samples tested within the triplex reaction are selected and populations individually identified and circled. Per plate, data from each assay were analyzed simultaneously according to individual assay format: duplex or triplex. The QX Manager Software relies upon the negative population to apply Poisson statistics to the results. These results were then analyzed for fractional abundance of the human genes in comparison to the mouse gene. Human gene fractional abundance as measured by hSRSF4 and hSF3A1 vs. mB2m was compared. If >10% difference between the two human genes is noted, reflex testing was initiated utilizing the hIPO8 gene. This gene is then compared to both human genes and a report was generated summarizing the results.

All assays were tested against tissues of murine origin known to harbor human cells. Each data point shown below represents a unique sample, but all assays can be compared along each sample's row. Samples were selected that varied widely in amount of human cellular presence.

Table 4 shows 10 samples of human-mouse mixed cell population test results for human amplicons. All human amplicons were compared the mouse amplicon and the overall concentration as measured by ddPCR and calculated fractional abundance of the human amplicon was reported. Samples 35 through 38 are human cells identified within mouse whole blood. The last three samples included are cultured human cells: HEK293 cells and HELA cells, followed by A9 (mouse) cultured cells.

TABLE 4

| | Concentration | | | | | |
|---|---|---|---|---|---|---|
| | (copies/μL) | | | Fractional Abundance (%) | | |
| Sample # | hSRSF4 | hIPO8 | hSF3A1 | hSRSF4 | hIPO8 | hSF3A1 |
| 25 | 313 | 263 | 467 | 33 | 29.2 | 38.1 |
| 26 | 847 | 684 | 1114 | 92 | 90.3 | 94 |
| 27 | 295 | 257 | 489 | 43.9 | 40.5 | 50.1 |
| 28 | 578 | 442 | 736 | 59.7 | 53.1 | 63.7 |
| 29 | 93.2 | 76.4 | 119 | 42.9 | 38.1 | 45.9 |
| 30 | 114 | 94 | 147 | 66.6 | 62.2 | 71.1 |
| 31 | 507 | 417 | 660 | 87.4 | 85 | 89.7 |
| 32 | 855 | 732 | 1135 | 57.4 | 53.5 | 62.9 |
| 33 | 862 | 771 | 1096 | 33 | 29.2 | 38.1 |
| 34 | 955 | 918 | 700 | 92 | 90.3 | 94 |
| 35 | 11.2 | 8.27 | 22.9 | 5.9 | 4.4 | 6.4 |
| 36 | 13.6 | 10.8 | 21.8 | 5.4 | 4.3 | 6.3 |
| 37 | 20.8 | 16.9 | 30.4 | 9.0 | 7.4 | 9.0 |
| 38 | 9.28 | 9.99 | 17.9 | 5.2 | 5.6 | 5.2 |
| HEK293 cells | 313 | 263 | 467 | 100 | 100 | 100 |
| HELA cells | 847 | 684 | 1114 | 100 | 100 | 100 |

| | |
|---|---|
| A9 (murine) | A9 cells test negative on all human assays and only show |
| cells | positive droplet signal within the mB2m HEX channel. |

This unique method of performing a triplex assay, including the direct comparison within the same well without the need for a standard curve, makes this a reliable and straightforward testing method.

1. A method of quantifying an amount of mouse cells and human cells in a cell sample, the method comprising:
(a) assembling a single polymerase chain reaction (PCR) assay comprising template nucleic acid molecules from the cell sample and reagents suitable for the amplification of: (i) nucleic acid molecules comprising one mouse locus, (ii) nucleic acid molecules comprising a first human locus, and (iii) nucleic acid molecules comprising a second human locus;
(b) partitioning the single PCR reaction assay into partitioned sections, wherein each partitioned section contains no target loci or a single locus, and wherein the locus can be amplified within the partitioned section;
(c) performing the single polymerase chain reaction assay;
(d) quantifying a number of partitioned sections having an amplification product corresponding to the mouse locus, an amplification product corresponding to the first human locus, and an amplification product corresponding to the second human locus; and
(e) determining a Poisson-modeled number of partitioned sections having an amplification product corresponding to the mouse locus, a Poisson-modeled number of partitioned sections having an amplification product corresponding to the first human locus, and a Poisson-modeled number of partitioned sections having an amplification product corresponding to the second human locus,
thereby determining an amount of mouse cells and human cells in the cell sample.

2. The method of claim 1, wherein if there is a discrepancy of at least about 10% between the Poisson-modeled number of partitioned sections having an amplification product corresponding to the first human locus and the Poisson-modeled number of partitioned sections having an amplification product corresponding to the second human locus, then
(e) assembling a single polymerase chain reaction (PCR) assay comprising template nucleic acid molecules from the sample and reagents suitable for the amplification of nucleic acid molecules comprising one mouse locus and a third human locus,
(f) partitioning the single PCR reaction assay into partitioned sections, wherein each partitioned section contains no target loci or most often a single locus, and wherein the locus can be amplified within the partitioned sections;
(g) performing the single polymerase chain reaction assay;
(h) quantifying a number of partitioned sections having an amplification product corresponding to the one mouse locus and the third human locus; and
(i) determining a Poisson-modeled number of partitioned sections having an amplification product corresponding to the one mouse locus and the third human locus, and averaging the number of partitioned sections from two out of the first human locus, the second human locus, and the third human locus in closest agreement.

3. The method of claim 1, wherein at least one of the human loci is stable in cancer.

4. The method of claim 1, wherein the mouse locus is from a beta-2 microglobulin (B2m) gene and wherein the first human locus and the second human locus is from a serine/arginine-rich splicing factor (SRFS4) gene, an importin 8 (IPO8) gene, or a splicing factor 3a protein complex (SF3A1) gene.

5. The method of claim 2, wherein the mouse locus is from a B2m gene and wherein the first human locus, the second human locus, and the third human locus are from a SRFS4 gene, an IPO8 gene, or a SF3A1 gene.

6. (canceled)

7. The method of claim 1, wherein the method further comprises amplifying and detecting two or more human short tandem repeats (STRs) of microsatellite regions.

8. (canceled)

9. The method of claim 1, wherein one or more Mycoplasma nucleic acid molecules are also amplified.

10. (canceled)

11. (canceled)

12. A method of quantifying an amount of mouse cells and human cells in a cell sample, the method comprising:
(a) assembling a single polymerase chain reaction (PCR) assay comprising template nucleic acid molecules from the sample and reagents suitable for the amplification of: (i) nucleic acid molecules comprising one mouse locus, (ii) nucleic acid molecules comprising a first human locus, (iii) nucleic acid molecules comprising a second human locus, and (iv) nucleic acid molecules comprising a third human locus;
(b) partitioning the single PCR reaction assay into partitioned sections, wherein each partitioned section contains no target loci or a single locus, and wherein the locus can be amplified within the partitioned section;
(c) performing the single polymerase chain reaction assay;
(d) quantifying a number of partitioned sections having an amplification product corresponding to the mouse locus, an amplification product corresponding to the first human locus, an amplification product corresponding to the second human locus; and an amplification product corresponding to the third human locus;
(e) determining a Poisson-modeled number of partitioned sections having an amplification product corresponding to the mouse locus, a Poisson-modeled number of partitioned sections having an amplification product corresponding to the first human locus, a Poisson-modeled number of partitioned sections having an amplification product corresponding to the second human locus, and a Poisson-modeled number of partitioned sections having an amplification product corresponding to the third human locus,
wherein if there is a discrepancy of at least about 10% between the Poisson-modeled number of partitioned sections having an amplification product corresponding to the first human locus and the Poisson-modeled number of partitioned sections having an amplification product corresponding to the second human locus, then determining a Poisson-modeled number of partitioned sections having an amplification product corresponding to the third human locus, and averaging the number of partitioned sections from two out of the first human locus, the second human locus, and the third human locus in closest agreement
thereby determining an amount of mouse cells and human cells in the cell sample.

13. A kit for determining the fractional abundance of human genomic nucleic acid molecules compared to mouse genomic nucleic acid molecules in a mixed cell sample comprising at least three sets of nucleic acid probes, primers, or pair of primers, wherein a first set of the at least three sets of nucleic acid probes, primers, or pair of primers is capable of specifically amplifying and detecting hSRSF4 nucleic acid molecules in a biological sample, a second set of the at least three nucleic acid sets of probes, primers, or pair of primers is capable of specifically amplifying and detecting hIPO8 nucleic acid molecules in the biological sample, and a third set of the at least three nucleic acid sets of probes, primers, or pair of primers is capable of specifically amplifying and detecting mB2m nucleic acid molecules in the biological sample.

14. The kit of claim 13, wherein the kit further comprises one or more of blocking agents, detectable labels or labeling agents, and reagents for hybridization.

15. The kit of claim 13, wherein the kit further comprises a fourth set of nucleic acid probes, primers, or pair of primers, wherein the fourth set of the at least three sets nucleic acid probes, primers, or pair of primers is capable of specifically amplifying and detecting hSF3A1 nucleic acid molecules in a biological sample.

16. The kit of claim 13, wherein the at least three sets of nucleic acid probes, primers, or pair of primers are appropriate for use in digital PCR.

17. (canceled)

18. (canceled)

19. The kit of claim 13, wherein the first set of nucleic acid probe, primer, or pair of primers, the second set of nucleic acid probe, primer, or pair of primers, and the third set of nucleic acid probe, primer, or pair of primers are each labeled with different detectable labels.

20. The kit of claim 19, wherein the first set of nucleic acid probe, primer, or pair of primers, the second set of nucleic acid probe, primer, or pair of primers, the third set of nucleic acid probe, primer, or pair of primers are each labeled with different fluorophores.

21. (canceled)

22. (canceled)

23. A method of quantifying an amount of mouse cells and human cells in a cell sample, the method comprising:
(a) assembling a single polymerase chain reaction (PCR) assay comprising template nucleic acid molecules from the cell sample and reagents suitable for the amplification of: (i) nucleic acid molecules comprising one mouse locus and
(ii) nucleic acid molecules comprising a first human locus;
(b) partitioning the single PCR reaction assay into partitioned sections, wherein each partitioned section contains no target loci or a single locus, and wherein the locus can be amplified within the partitioned section;
(c) performing the single polymerase chain reaction assay;
(d) quantifying a number of partitioned sections having an amplification product corresponding to the mouse locus and an amplification product corresponding to the first human locus; and
(e) determining a Poisson-modeled number of partitioned sections having an amplification product corresponding to the mouse locus and a Poisson-modeled number of partitioned sections having an amplification product corresponding to the first human locus,
thereby determining an amount of mouse cells and human cells in the cell sample.

24. The method of claim 23, wherein the human loci is stable in cancer.

25. The method of claim 23, wherein the mouse locus is from a beta-2 microglobulin (B2m) gene and wherein the first human locus is from a serine/arginine-rich splicing factor (SRFS4) gene, an importin 8 (IPO8) gene, or a splicing factor 3a protein complex (SF3A1) gene.

26. (canceled)

27. The method of claim 23, wherein the method further comprises amplifying and detecting two or more human short tandem repeats (STRs) of microsatellite regions.

28. The method of claim 27, wherein the two or more human STRs are detected in two or more loci comprising D3S1358, TH01, D21S11, D18S51, Penta E, D5S818, D13S317, D7S820, D16S539, CSF1PO, Penta D, Amelogenin, vWA, D8S1179, TPOX, FGA, D19S433, D2S1338, or combinations thereof.

29. The method of claim 23, wherein one or more Mycoplasma nucleic acid molecules are also amplified.

30. (canceled)

31. (canceled)
